(19) United States
(12) Patent Application Publication
(10) Pub. No.: US 2024/0156714 A1
(43) Pub. Date: May 16, 2024

(54) SOLID AVOCADO OIL RICH IN PALMITIC ACID

(71) Applicant: GREENTECH, Saint Beauzire (FR)

(72) Inventor: Antoine PICCIRILLI, Poitiers (FR)

(73) Assignee: GREENTECH, Saint Beauzire (FR)

(21) Appl. No.: 18/285,336

(22) PCT Filed: Mar. 31, 2022
(86) PCT No.: PCT/EP2022/058720

§ 371 Date: Oct. 2, 2023

(30) Foreign Application Priority Data
Mar. 31, 2021 (FR) FR2103333

(51) Int. Cl. A61K 8/92; A23D 9/04; A23G 1/36; A61K 8/36; A61Q 1/04; A61Q 15/00; A61Q 19/00

(52) U.S. Cl. CPC A61K 8/922; A23D 9/04; A23G 1/36; A61K 8/361; A61Q 1/04; A61Q 15/00; A61Q 19/00

(57) ABSTRACT

A solid avocado oil is characterized in that its palmitic acid content in relation to total fatty acids is between 15 and 35% and its complete melting point is within a range of from 20 to 50° C., the avocado oil being obtained from avocado of a variety selected from the list consisting of Hass, Fuerte, Bacon, Pinkerton, Ettinger, Lula, Ryan, Barker, and/or Peruano. A cosmetic or pharmaceutical composition is characterized in that it comprises, in a cosmetically or pharmaceutically acceptable vehicle, a solid avocado oil according to the invention. A method for obtaining a solid avocado oil is characterized in that it is carried out on fresh or dehydrated fruit, crude avocado oils, or co-products from the refining or dewaxing of crude avocado oils.

The present invention relates to a solid avocado oil which is characterized in that it has a palmitic acid content which is significantly higher than that of conventional avocado oil. The invention also relates to the use of said oil in the fields of cosmetics, pharmaceuticals, and foods.

Virgin or refined avocado oil obtained by cold pressing or by water extraction (the so-called centrifugal method) is an oil that is commonly used in cosmetics for its nutritional and emollient properties and for its ability to penetrate into the upper layers of the epidermis. It is also valued in cosmetics for its good relative stability against oxidation and its natural vitamin E content (alpha-tocopherol (Woolf et al., Gourmet & Health-Promoting Specialty Oils, pp. 73-125, edited by Robert A. Moreau and Ataf Kamal-Eldin. AOCS Press 2009).

More recently, avocado oil has undergone spectacular development in human nutrition, particularly in the United States and the United Kingdom. Highly appreciated for the health benefits linked to its high content of monounsaturated fatty acids and antioxidants, it has now entered into direct competition with olive oil (Hilary S. First Report on Quality and Purity of Avocado Oil Sold in the US. Food Control 116 (2020) 107328). Furthermore, in terms of taste, avocado oil has a very slightly buttery aroma and is devoid of the often strong notes of first cold-pressed olive oils.

Its annual global production is now estimated at 160,000 tons, 50% of which is produced in Mexico, the world's leading producer of avocado, with more than 1.5 million tons of fruit harvested each year (Fernandes et al. Chemical Characterization of Commercial and Single-Variety Avocado Oils. Grasa y aceites, vol. 69, no. 2 (2018)).

Avocado oil is actually a co-product of fruit production. It is in fact obtained from fruits that have been downgraded for reasons of noncompliance with consumer market criteria (appearance, freshness, size, etc.). Consequently, fruits that are too small, dented, or affected by diseases associated with attacks by pests or fungi are directly dedicated to the production of oil (South African Avocado Growers' Association Yearbook 1987. 10: 159-162. TP Human. Proceedings of the First World Avocado Congress. Oil as a Byproduct of the Avocado).

Avocado oil is extracted from the pulp of the previously pitted fruit. The avocado (Persea americana Mill) is a fruit that is described as oil-bearing on account of its pulp being very rich in oil (>15%) and water (60%). Taxonomically, we distinguish between the Mexican, Guatemalan, and West Indian landraces. The primary varieties resulting from crosses of these landraces—and those most cultivated today—are the Hass and Fuerte avocados. In Mexico, more than 90% of production is of the Hass variety. This variety is known to be the most concentrated in pulp oil (greater than 20%). Other varieties that are grown around the world (Chile, Peru, South Africa, Kenya, New Zealand, etc.) include the Bacon, Pinkerton, Ettinger, Lula, Ryan, Barker, and Peruano varieties.

In terms of production processes, we distinguish between first-pressed avocado oils which are chiefly intended for specialized nutrition and refined oils for the cosmetics and everyday food markets. Pressed oils are obtained using a process similar to that used for olive oil. This entails extraction consisting in treating the (previously peeled and pitted) fruit pulp with hot water, followed by centrifugation to separate the water and the fat (Costagli et al. Journal of Agricultural Engineering, 2015, vol. XL VI: 467, pp. 115-122).

Refined avocado oils are obtained from crude avocado oils, and refining consists of chemical neutralization with soda, acid degumming, winterization, and bleaching on bleaching earth, followed finally by deodorization under a vacuum.

In terms of its fatty acid composition and all varieties combined, unrefined pulp oil is primarily made up of oleic acid (55 to 68%), palmitic acid (13 to 22%), and linoleic acid (9 to 15%). Also note the presence of palmitoleic acid at a level of 4 to 10%. Likewise, refined avocado oils obtained from the Hass variety, which are those most used particularly in cosmetics and consumer foods, have rather middling contents of oleic, palmitic, and linoleic acids, approaching 60, 12, and 10%, respectively. Since the aim of refining is to offer clear oils which do not cloud in the cold with a minimum cold resistance of 5.5 hours at 0° C., it in fact results in substantial losses of fatty acids and, in particular, palmitic acid.

In cosmetics, palmitic acid is of great interest for the skin. Indeed, palmitic acid plays an essential role in the synthesis of ceramides of the stratum corneum, particularly ceramides 1 and 2. These ceramides ensure the integrity of the epidermis and the functionality of the hydrolipid film in terms of hydration and maintenance of epidermal homeostasis (J. McIntosh. Organization of skin stratum corneum extracellular matrix lamellae: Diffraction evidence for asymmetric distribution of cholesterol. Biophysical Journal, vol. 85, September 2003: 1675-1681).

Likewise, palmitic acid is directly involved in the morphogenesis of the epidermis and the skin barrier effect function (Mieremet et al. Contribution of palmitic acid to epidermal morphogenesis and lipid barrier function in human skin equivalents. International of Molecular Sciences. Int. J. Mol. Sci. 2019, 20, 6069. pp. 1-18).

Palmitic acid is an essential fatty acid that makes up hair lipids. It is in fact the most abundant acid in the hair fiber, with a content of 2760 μg/g of hair (Sang-Sun Song et al. Prevention of lipids from hair by surface and internal modification. Nature Scientific Reports, (2019), 9-9834). This palmitic acid content is greatly reduced by successive shampooing, which tends to degrade the quality of the fiber. Also, it is essential to be able to nourish the hair with a regular supply of palmitic acid via hair care products. Likewise, palmitic acid is also a constituent of essential ceramides in hair. These ceramides play an essential role in maintaining and protecting the hair fiber against external aggressions such as UV, pollution, and chemical coloring. Palmitic acid is therefore one of the essential nutrients for preventing alopecia.

Nutritionally, an intimate relationship exists between the adipocytes which constitute the fatty tissues of the skin and the hair follicles of the hair. Indeed, many growth factors interact in the adipocyte-follicle relationship. The hair follicle is thus surrounded by a macro-environment made up of fundamental lipids, among which there is an abundance of palmitic acid, which is also one of the major acids constituting the energy reserves of the adipocyte (Schmidt et al. Unraveling hair follicle-adipocyte communication. Exp Dermatol 2012 November 21(11): 827-830). The fatty acids that are provided by food and stored as an energy reserve in adipocytes thus contribute to the development of the hair follicle, which is the central organ in the synthesis of hair fiber. Adequate and suitable nutritional intake may advantageously contribute to the development of healthy hair.

It is also known that palmitic acid intervenes in a crucial biological respect, since it makes an essential step in cell renewal possible through the palmitoylation of the proteins constituting cell walls (Linder, ME, “Reversible modification of proteins with thioester-linked fatty acids,” Protein Lipidation, F. Tamanoi and DS Sigman, eds., pp. 215-40 (San Diego, CA: Academic Press, 2000).

Indeed, carbohydrates (sugars) are transformed in vivo into palmitic acid, which is then stored in adipocytes. While we encourage limiting the consumption of sugars in the prevention of diabetes, controlled nutritional intake of bioavailable palmitic acid represents an interesting way to produce reserve lipids without utilizing the sugar metabolic pathway.

Finally, the main dietary intake of palmitic acid is through the consumption of palm oil, meat, and dairy products. However, these foods are currently suspected of being the cause of obesity problems and cardiovascular diseases. What is more, palm oil, which is highly criticized for environmental reasons in both food and cosmetics, tends to be replaced by more sustainable oils.

In this context, the supply of palmitic acid via a plant source that is more environmentally friendly and allows for the joint provision of monounsaturated fatty acids is not currently satisfied.

Application CA 2 628 950 describes a method for obtaining an avocado oil that is rich in triglycerides. The method consists in producing 2 oils by means of a molecular distillation process: a distillate and a heavy oil corresponding to the distillation residue fraction. After conventional refining, it appears that the oil resulting from the distillation residue fraction, called the heavy fraction, is rich in sterols and devoid of unsaponifiables (such as acetogenins and furan lipids) and partial glycerides (mono- and diglycerides) compared to an oil that has not undergone molecular distillation. However, it should be noted that the oil with which the invention is concerned is depleted of palmitic acid compared to the oil of reference.

Application PCT/FR01/00814 describes a method for concentrating unsaponifiables in vegetable oils, among which avocado oil is mentioned. The concentration of unsaponifiables describes a molecular distillation process which leads to an oil that is concentrated in sterols and fat-soluble vitamins as well as a residue oil (heavy fraction) that is depleted of unsaponifiables. However, this process does not result in an oil that is rich in palmitic acid.

The article Yanty NAM et al.: “Effect of varietal differences on composition and thermal characteristics of avocado oil,” JOURNAL OF THE AMERICAN OIL CHEMISTS' SOCIETY, SPRINGER-VERLAG, BERLIN/HEIDELBERG, vol. 88, no. 12, Jun. 17, 2011 (2011-06-17), pp. 1997-2003, XP019975762, ISSN: 1558-9331, DOI: 10.1007/511746-011-1877-X describes a comparison between oils from Malaysian avocados and oils from Hass-variety avocados. These two oils are obtained by extraction with petroleum ether. It should be noted that avocado oils of Malaysian origin are not obtained from the avocado varieties Hass, Fuerte, Bacon, Pinkerton, Ettinger, Lula, Ryan, Barker, and/or Peruano. The Hass-variety avocado oils cited are not solid.

Application US 2005/058731 describes an antimicrobial composition comprising avocado oil but does not disclose a specific avocado oil or the palmitic acid content or the melting point thereof.

Application EP 1 139 771 describes a food product comprising avocado oil but does not disclose a solid avocado oil or the palmitic acid content or melting point thereof.

The article Yanty N. et al.: “Effect of fractional crystallization on composition and thermal characteristics of avocado (Persea americana) butter,” Journal of Thermal Analysis and calorimetry, Nov. 25, 2011 (2011-11-25), pp. 2203-2209, XP055863496, Dordrecht DOI: 10.1007/s10973-011-2055-y describes a process for extracting avocado oil from avocado of a Malaysian avocado variety. This process includes extraction with petroleum ether, mixing with acetone, and successive crystallization/filtration steps. However, the process does not include a winterization step. Following the first crystallization/filtration step, the precipitated fraction, termed AVS, has a palmitic acid content well above 35% compared to the total triglyceride content. It should also be noted that these oils are not obtained from Hass, Fuerte, Bacon, Pinkerton, Ettinger, Lula, Ryan, Barker, and/or Peruano avocado varieties.

Certain expressions or words may be used in this application to indicate technical elements, facts, properties, or characteristics. Their meaning is given below:

Triglycerides are glycerol esters in which the 3 hydroxyl groups of glycerol are esterified with fatty acids.

Their general formula is as follows:

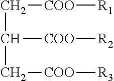

R1, R2, and R3 being identical or different saturated or unsaturated alkyl chains. If at least one of R₁, R₂, and/or R₃ is a saturated alkyl chain with 16 carbon atoms (C16:0), this means that the hydroxyl group is esterified with palmitic acid. If at least one of R₁, R₂, and/or R₃ is an alkyl chain with 16 unsaturated carbon atoms (C16:1), this means that the hydroxyl group is esterified with palmitoleic acid.

If at least one of R₁, R₂, and/or R₃ is a saturated alkyl chain with 18 carbon atoms (C18:0), this means that the hydroxyl group is esterified with stearic acid. If at least one of R₁, R₂, and/or R₃ is an alkyl chain with 18 unsaturated carbon atoms (C18:1), this means that the hydroxyl group is esterified with oleic acid. If at least one of R₁, R₂, and/or R₃ is an alkyl chain with 18 unsaturated carbon atoms (C18:2), this means that the hydroxyl group is esterified with linoleic acid.

Virgin oil is obtained by cold-pressing the fruit or fruit pulp.

Oil is understood to mean an oil that is obtained after an extraction consisting of treating the (previously peeled and pitted) fruit pulp with hot water, followed by centrifugation to separate the water and the fat.

Crude oil is understood to mean virgin oil or avocado oil that has not undergone a refining process.

Refined oil is understood to mean a virgin oil or an avocado oil that has undergone a refining process.

The process of refining a vegetable oil is a well-known process as described, for example, in application CA2628950.

The method comprises the following steps:
acid treatment
a neutralization step
winterization or dewaxing
a bleaching step
a deodorization step.

The acid treatment is preferably acid conditioning, making it possible to carry out degumming and eliminate phospholipids. Acid treatment is typically carried out using a weak acid such as phosphoric acid or citric acid or using a strong acid such as sulfuric acid or hydrochloric acid. Generally, the acid treatment is carried out under stirring at a temperature between 30 and 70° C. Other processes may be preferred, such as microfiltration, which is a low-pressure membrane process that is used for the filtration of colloids, or processes that employ biotechnology, such as enzymatic degumming.

Chemical neutralization, which typically follows the acid treatment, eliminates free fatty acids, rids the oil of phospholipids which have undergone the conditioning operation, eliminates metallic traces, and facilitates bleaching by destroying a certain number of pigments and colored compounds of oxidative origin. Chemical neutralization is typically carried out using a basic agent such as sodium hydroxide, potassium carbonate, or a tertiary amine (DMHA). The fatty acids are separated out by centrifugation or filtration into the form of soaps which also contain the mucilages and various impurities eliminated during this treatment.

Bleaching, which follows chemical neutralization, generally makes it possible to eliminate the colored pigments that are only partially destroyed by neutralization. Bleaching is typically carried out using bleaching earths and/or charcoal, advantageously until the avocado oil has a light or very light color.

The bleaching earths used are natural montmorillonite-type clays consisting primarily of calcium and magnesium aluminosilicates and activated by acid treatment. The activated carbons used can be obtained from peat, wood, lignite, coal, or coconut husks. These products are typically activated at high temperature, either by steam or by means of a chemical process.

The refrigeration or winterization step, which follows bleaching, can be carried out at a temperature between 5 and 18° C., advantageously for 1 to 15 days. Typically, refined avocado oil is cooled gradually, preferably under slow stirring, to a temperature of around 10 to 12° C. Generally, the oil is then maintained at this temperature for 48 hours and is then filtered under pressure. Refrigeration precipitates triglycerides rich in saturated fatty acids.

Deodorization, which follows refrigeration, generally eliminates and extracts volatile compounds and malodorous molecules as well as various contaminants such as pesticides and polycyclic aromatic hydrocarbons. Deodorization is typically carried out at a temperature between 150 and 210° C., advantageously under a vacuum, generally under a stream of saturated steam or nitrogen. Deodorization can, for example, be carried out under a pressure of between 2 and 20 mmHg. In particular, deodorization can be carried out at a temperature of around 180 to 200° C. under a pressure of around 4 to 6 mmHg.

A semi-refined oil is an oil that has undergone partial refining (without the winterization and deodorization steps).

A solid material at 20° C. is understood to mean an oil having a pour point as determined by the ASTM D 97 method of between 8 and 15° C.

The invention relates to a solid avocado oil which is characterized in that its content of palmitic acid relative to total fatty acids is within a range of from 15 to 35% and its full melting point is within a range of from 20 to 50° C., the avocado oil being obtained from avocado varieties selected from the list consisting of Hass, Fuerte, Bacon, Pinkerton, Ettinger, Lula, Ryan, Barker, and/or Peruano.

In one embodiment, the avocado is a Hass-, and/or Fuerte-variety avocado.

In one embodiment the avocado is a Hass-variety avocado.

According to one preferred embodiment of the invention, the solid avocado oil is not obtained from avocado of ++96 variety of Malaysian origin, particularly as defined in Yanty, N. A. M., Marikkar, J. M. N. & Long, K. Effect of varietal differences on composition and thermal characteristics of avocado oil. J Am Oil Chem Soc 88, 1997-2003 (2011).

It also relates to a solid avocado oil which is characterized in that its palmitic acid content relative to total fatty acids is within a range of from 16 to 32%, and its complete melting point is within a range of from 20 to 50° C.

It also relates to a solid avocado oil which is characterized in that its palmitic acid content relative to total fatty acids is within a range of from 20 to 30%, and its complete melting point is within a range of from 20 to 50° C.

In one embodiment, the complete melting point is within a range of from 23.0 to 40.0° C.

In one embodiment, the complete melting point is within a range of from 25.0 to 35.0° C.

The solid avocado oil according to the invention has a pour point as determined by the ASTM D 97 method of between 8 and 15° C.

It also relates to a method which utilizes a solid avocado oil according to the invention as a source of palmitic acid.

The solid avocado oil according to the invention has a palmitic acid content which is increased by more than 25 to 75% compared to the starting raw material, preferably by more than 30 to 60%.

The solid avocado oil according to the invention, which is preferably obtained from Hass-variety avocado, has a pour point as determined by the ASTM D 97 method of between 8 and 15° C.

It also relates to a method which utilizes a solid avocado oil according to the invention as a source of palmitic acid, the solid avocado oil being preferably obtained from Hass-variety avocado.

The solid avocado oil according to the invention, which is preferably obtained from Hass-variety avocado, has a palmitic acid content which is increased by more than 25 to 75% compared to the starting raw material, preferably by more than 30 to 60%.

In one embodiment, the palmitic acid content is increased by at least 40% compared to the starting raw material.

In one embodiment, the palmitic acid content is increased by at least 50% compared to the starting raw material.

For the purposes of the present invention, the term “starting raw material” means an avocado oil obtained by extraction with petroleum ether, particularly according to the protocol as described in the article Yanty NAM et al., “Effect of varietal differences on composition and thermal characteristics of avocado oil,” JOURNAL OF THE AMERICAN OIL CHEMISTS' SOCIETY, SPRINGER-VERLAG, BERLIN/HEIDELBERG, vol. 88, no. 12, Jun. 17, 2011 (2011-06-17), pp. 1997-2003, XP019975762, ISSN: 1558-9331, DOI: 10.1007/S11746-011-1877-X, or a crude avocado oil which can alternatively be obtained either through extraction consisting in treating the (previously peeled and pitted) fruit pulp with hot water, followed by centrifugation to separate water and fat or a crude virgin oil which is obtained by cold pressing.

In a particular embodiment of the invention in which the solid avocado oil according to the invention is obtained from co-products resulting from the refining or dewaxing of crude avocado oils, such as by example winterization residues, the solid avocado oil according to the invention is characterized by a palmitic acid content which is increased by more than 25 to 75% compared to refined avocado oil, preferably by more than 30 to 60%.

The solid avocado oil according to the invention has a cold resistance at 0° C. (273 K) of less than 2.0 hours.

In one embodiment, the solid avocado oil according to the invention has a cold resistance at 0° C. (273 K) of less than 1.5 hours.

In one embodiment, the solid avocado oil according to the invention has a cold resistance at 0° C. (273 K) of less than 1.0 hours.

Cold resistance is understood to mean the time from which an oil becomes cloudy when kept at a temperature of 0° C. under the conditions as described in the so-called AOCS Cold Test method (Cc 11-53).

The invention also relates to a solid avocado oil as defined above which is characterized in that, in differential thermal analysis, it exhibits a majority peak of slow melting a majority peak of slow melting falling in a range of from 30.0 to 50.0° C.

The invention also relates to a solid avocado oil as defined above which is characterized in that its content of triglycerides in which palmitic acid is in position 2 of the glycerol is greater than or equal to 10% relative to the total triglyceride content.

The invention also relates to a solid avocado oil as defined above which is characterized in that its relative content of palmitoleic acid vector triglycerides is greater than or equal to 4.5% relative to the total triglyceride content. As such, solid avocado oil is a source of palmitoleic acid.

Biologically, palmitoleic acid is a fatty acid that constitutes glycerides stored in human adipose tissue. It is produced by biosynthesis from palmitic acid under the action of the enzyme stearoyl-CoA desaturase-1 (A9 desaturase). It is known for its anti-inflammatory and PPAR-alpha activating activity (Vannice G K et al. (2018). Is palmitoleic acid a plausible nonpharmacological strategy to prevent or control chronic metabolic and inflammatory disorders? Molecular Nutrition & Food Research: 62 (1), 1700504).

Palmitoleic acid is also the most abundant monoenic acid that is present in sebum and which also has significant antimicrobial activity (. J J Wille et al. Palmitoleic acid isomer (C16:1delta6) in human skin sebum is effective against gram-positive bacteria. Skin Pharmacol Appl Skin Physiol. May-June 2003; 16(3), 176-87).

It also relates to a method which utilizes a solid avocado oil according to the invention as a source of palmitoleic acid.

The invention also relates to a solid avocado oil as defined above which is characterized in that its relative triolein content is less than or equal to 25%, preferably less than or equal to 23%, relative to the total triglyceride content..

The solid avocado oil according to the invention is characterized in that, in differential thermal analysis, it exhibits a majority peak of slow melting between 30.0 and 50.0° C. Consequently, solid avocado oil is a soft solid fat at 25° C. unlike virgin or refined avocado oils, which are completely fluid at 25° C. and do not exhibit a majority peak of slow melting in differential thermal analysis of between 30.0 and 50.0° C.

According to one advantageous embodiment of the invention, the solid avocado oil according to the invention is characterized in that it is obtained by extraction in the absence of organic solvent, preferably in the absence of solvent.

For the purposes of the present invention, the term “organic solvent” refers to any solvents comprising organic compounds which contain carbon atoms, particularly solvents of petroleum origin.

According to one particularly preferred embodiment, the solid avocado oil according to the invention is characterized in that it is obtained by extraction in the absence of petroleum ether.

According to one advantageous embodiment of the invention, the solid avocado oil according to the invention is characterized in that it is obtained by means of at least one winterization step.

According to one particularly preferred embodiment of the invention, the solid avocado oil according to the invention is characterized in that it is obtained by means of at least one winterization step and in absence of organic solvent, preferably in the absence of solvent.

The solid avocado oil according to the invention, which is preferably obtained from Hass-variety avocado, can be obtained from fresh or dehydrated fruits as starting vegetable raw materials.

The solid avocado oil according to the invention, which is preferably obtained from Hass-variety avocado, can also be obtained from crude avocado oils from fresh or dehydrated fruits. Crude oils can be obtained by cold pressing, by extraction using a solvent or supercritical fluids, by aqueous extraction particularly using the so-called centrifugal method, or even enzyme-assisted aqueous extraction.

The solid avocado oil according to the invention, which is preferably obtained from Hass-variety avocado, can also be obtained from co-products resulting from the refining or dewaxing of crude avocado oils, such as residues of winterization, for example.

Preferably, the solid avocado oil is prepared from winterization residues resulting from the dewaxing of crude avocado oils, virgin oils, semi-refined oils, or even refined oils.

The solid avocado oil according to the invention, which is preferably obtained from Hass-variety avocado, is refined according to a process identical to those implemented in the field of vegetable oils. A refining process is understood to mean a physical or chemical refining process. Refining includes all of the known steps or at least one of them. These steps are as follows:
1) Degumming with water, acid, or in the presence of an enzyme;
2) chemical neutralization (with soda or potash);
3) physical neutralization by distillation under high vacuum or by deodorization under a vacuum in the presence of a vector (water vapor, nitrogen, carbon dioxide);
4) bleaching on bleaching earth (in the presence or absence of activated clay) and/or in the presence of activated carbon, with or without silicate;
5) cold crystallization in the presence or absence of a solvent;
6) deodorization and/or vacuum distillation, neutralizing or not, with or without a vector (water vapor, nitrogen, carbon dioxide).

As will readily be understood, the refining steps can be carried out in the order that is most suitable for carrying out the invention as a function of the nature and composition criteria of the avocado oily raw material used to produce the solid avocado oil according to the invention.

In one preferred embodiment of the invention, the solid avocado oil can undergo a second or a plurality of successive cold crystallization steps in order to increase the palmitic acid content.

The invention relates to a method for obtaining a solid avocado oil, preferably from Hass-variety avocado, which is characterized in that it is carried out from:
fresh or dehydrated fruit,
crude avocado oils, or
co-products from the refining or dewaxing of crude avocado oils.

When the method for obtaining a solid avocado oil is carried out on co-products resulting from the refining or dewaxing of crude avocado oils, preferably winterization residues, or when it comprises a step of using co-products from the refining or dewaxing of crude avocado oils, preferably winterization residues, it has the advantage of using a conventionally non-valued co-product of the avocado oil preparation process.

The solid oil obtained in this manner is therefore of significant interest from an ecological and environmental point of view.

The invention also relates to a method for obtaining a solid avocado oil as described previously which is characterized in that it is carried out on:
fresh or dehydrated fruit,
   and that it comprises at least the steps of:
grinding, extraction of crude oil,
eliminating a fluid oil by winterization and filtration,
obtaining a solid fat,
deodorization.

The invention also relates to a method for obtaining a solid avocado oil as described previously which is characterized in that it is carried out on:
crude avocado oil,
   and that it comprises at least the steps of:
eliminating a fluid oil by winterization and filtration,
obtaining a solid fat,
deodorization.

The invention also relates to a method for obtaining a solid avocado oil, preferably from Hass-variety avocado as described previously, which is characterized in that it is carried out on:
fresh or dehydrated fruit,
   and that it comprises at least the steps of:
grinding, extraction of crude oil,
eliminating a fluid oil by winterization and filtration,
obtaining a solid fat,
deodorization.

The invention also relates to a method for obtaining a solid avocado oil, preferably from Hass-variety avocado as described previously, which is characterized in that it is carried out on:
crude avocado oil,
   and that it comprises at least the steps of:
eliminating a fluid oil by winterization and filtration,
obtaining a solid fat,
deodorization.

The invention also relates to a method for obtaining a solid avocado oil as described previously which is characterized in that it is carried out on:
co-products from the refining or dewaxing of crude avocado oils,
   and that it comprises at least the steps of:
deodorization.

According to one embodiment of the invention, the method for obtaining a solid avocado oil, preferably from Hass-variety avocado as described previously, is characterized in that it is carried out on:
co-products from the refining or dewaxing of crude avocado oils, and that it comprises at least the steps of:
deodorization,
   the co-products resulting from the refining or dewaxing of crude avocado oils being selected from the list consisting of winterization residues.

The invention also relates to a method of obtainment as described previously which is characterized in that, before the deodorization step, it comprises at least one of the following steps:
degumming
chemical neutralization
physical neutralization
bleaching
cold crystallization and isolation of the precipitated fraction.

The invention also relates to a method of obtainment as described previously which is characterized in that it further comprises a step of fractionation by hydrolysis.

The solid avocado oil according to the invention is in the form of a solid raw material at 20° C. having emollient and spreading properties, particularly spreadability.

The solid avocado oil according to the invention is characterized in that it is easily formulated for the preparation of cosmetic and food products.

The invention also relates to a cosmetic or pharmaceutical composition which is characterized in that it comprises, in a cosmetically or pharmaceutically acceptable vehicle, a solid avocado oil according to the invention.

In one embodiment, the cosmetic or pharmaceutical composition according to the invention is characterized in that it further comprises an active ingredient.

The solid avocado oil according to the invention is intended to be incorporated into cosmetic products as an active ingredient or functional raw material.

Therefore, the invention also relates to the use of the solid oil according to the invention for the preparation of cosmetic and/or nutraceutical compositions.

Therefore, the invention also relates to the use of the solid oil according to the invention, preferably from Hass-variety avocado, for the preparation of cosmetic and/or nutraceutical compositions.

A cosmetic product is understood to be any substance or mixture that is intended to be brought into contact with the healthy superficial parts of the human body (healthy epidermis, healthy hair and capillary systems, healthy nails, healthy lips, and healthy external genitalia) or with healthy teeth and healthy oral mucous membranes with the aim, exclusively or primarily, of cleaning them, perfuming them, modifying their appearance, protecting them, maintaining them in good condition, or correcting body odors (Article 2 Cosmetic Regulations and Article L.5131-1 Public Health Code (CSP).

The solid avocado oil according to the invention can be incorporated into the different categories of cosmetic products, including products for healthy skin, hygiene products, hair products, depilatory products, products for nail care and adornment.

Products for healthy skin include creams, emulsions, lotions, gels, and oils for the skin, beauty masks, liquid foundations, pastes, and/or powders, makeup powders, powders for application after bathing, powders for personal hygiene, preparations for baths and showers, particularly selected from among salts, foams, oils, and/or gels, sun care products, sunless tanning products, products for lightening the skin, anti-wrinkle products, shaving products, particularly selected from among soaps, foams, and/or lotions, makeup and makeup removal products, and products intended to be applied to the lips.

Hygiene products are understood to mean soaps toiletries, deodorant soaps, dental and oral hygiene products, external intimate hygiene products, deodorants, and antiperspirants.

Hair products are understood to mean hair dyes, products for waving, straightening, and fixing hair, styling products, cleaning products for hair, particularly selected from among lotions, powders, and/or shampoos, maintenance products for hair, particularly selected from among lotions, creams, and/or oils, styling products, particularly selected from among lotions, hair sprays, and/or pomades.

The solid avocado oil according to the invention is incorporated into a physiologically, cosmetologically, and dermatologically acceptable composition. This composition may be aqueous, hydroalcoholic, or even comprise an aqueous phase that is combined or not combined with an oily phase.

Intended for application to the skin and hair, the composition containing the solid avocado oil according to the invention may be a single or triple emulsion which may or may not contain liposomes, niosomes, or even oleosomes. In terms of droplet size, emulsions can be nano- or microemulsions.

The compositions for skin and hair may contain moisturizing and humectant agents selected in a non-limiting manner from the group consisting of alpha-, beta-, gamma-hydroxy acids (particularly lactic, malic, citric, glycolic, tartaric, gluconic acids), sodium-2 pyrrolidone, mucopolysaccharides, proteoglycans, glycoaminoglycans, glucans, glycols (glycerol, polyglycerols, propylene glycol, 1,3 propanediol, pentanediol, hexanediol), hyaluronic acid, hydrolysates of proteins, soluble collagens, urea, D-panthenol, vitamin E esters (linoleate, acetate), lanolin, and derivatives thereof.

Compositions for skin and hair may contain hydrophilic and/or lipophilic emollients belonging in a non-limiting manner to the group consisting of vegetable oils, synthetic triglycerides (medium-chain triglycerides, for example), vegetable and synthetic butters, hydrogenated oils, benzoic acid esters, mineral oils, linear and branched hydrocarbons of mineral or vegetable origin, ethers, fatty acid esters, synthetic fatty alcohols and those of vegetable origin, Guerbet alcohols, glycol esters (propylene glycol di-caprylate/caprate, for example), polyethylene and propylene glycol esters, silicones (particularly volatile cyclomethicones, linear polysiloxanes such as dimethicones, and alkyl or phenyl trimethicones).

Compositions for skin and hair may contain thickening agents which may belong in a non-limiting manner to the group consisting of mineral, vegetable, and synthetic waxes (example: carnauba, candelilla, sunflower wax), hydrogenated glycerides, fatty acids, fatty alcohols, cellulose derivatives (particularly methyl, ethyl, and propyl cellulose, carboxymethyl cellulose), starch and derivatives thereof (dextrose, dextrins, sodium polyacrylate starch, aluminum octylsuccinate), natural gums (arabic, guar, carob, tragacanth), pectins, agar, alginates, carrageenans, xanthan gums, gelatin, mineral oxides (silica, bentonite, aluminosilicates), aluminum and magnesium stearates, zinc stearate, crosslinked or non-crosslinked carbomers, C₁₀-C₃₀ alkyl acrylates, polyolefins such as polybutenes and polydecenes, polyethylene and polypropylene, ethoxylated or propyloxylated alcohols and fatty acids, polyacrylam ides, copolymers of fatty acid dimers/polyols, or even fatty alcohol/diacid dimers.

Compositions intended for skin and hair in the form of emulsions contain emulsifying surfactants in a known manner. Without limitation, these emulsifiers belong to the group consisting of cationic, anionic, nonionic, and amphoteric surfactants. These agents include, inter alia, fatty acid soaps, alkyl sulfates, alkyl ether sulfates, alkyl sulfonates, alkyl and olefin sulfonates, sulfosuccinates, acyl isothionates, sulfated alkanolamides, imidazole sulfates, taurine and methyl taurine sulfonates, alkylphosphates, quaternary ammoniums, ethoxylated amines, imidazolines, alkylmamines, lecithins, ethoxylated fatty alcohols and phenyl alcohols, sucroesters (of sorbitol, glucose, sucrose), oxyethylenated sugars, alkyl polyglucosides (APG), monoglycerides, polyglycerol esters, and dimethicone copolyols.

Compositions intended for skin and hair can also be sun protection products. Consequently, the sun care compositions contain, in a non-limiting manner, chemical filters and mineral filters. These filters we find, in a non-limiting manner, UV-B and UV-A filters such as octyl methyl cinnamate, methyl benzylidene camphor, phenylbenzimidazole sulfonic acid, octyl triazole, avobenzone, octyl salicylate, octyl dimethyl paba, octocrylene, benzophenones, methyl anthralinate, and zinc and titanium oxides.

Finally, compositions intended for skin and hair may contain conventional agents such as active ingredients, preservatives, perfumes, pigments, complexing agents, anti-odor agents, antiperspirants, antioxidants, and/or solvents (alcohols, glycols).

As an active ingredient, the solid avocado oil according to the invention is incorporated as a cosmetic treatment agent for healthy keratin fibers (healthy skin, healthy hair, healthy nails, healthy eyelashes).

The invention therefore also relates to the use of the solid oil according to the invention as an active ingredient for the cosmetic treatment of healthy skin and/or healthy keratin fibers, particularly healthy hair, healthy nails, and/or healthy eyelashes.

The invention therefore also relates to the use of the solid oil according to the invention, preferably from Hass-variety avocado, as an active ingredient for the cosmetic treatment of healthy skin and/or keratin fibers, particularly healthy hair, healthy nails, and/or healthy eyelashes.

More particularly, solid avocado oil is a lipid-replenishing, nutritious, moisturizing agent that is constitutive of the hydrolipidic film but is also used as a promoter of the synthesis of epidermal lipids and/or as an agent for treating mature skins, winter xerosis, atopy, eczema, alopecia, dandruff, or even keratin fibers damaged by UV rays or aggressive substances (shampoos, substances used in hair coloring, medications, etc.).

The invention therefore relates to the cosmetic use of the solid oil according to the invention as a lipid-replenishing, nourishing, anti-dandruff, and/or moisturizing agent for healthy skin, particularly healthy scalp, and/or for the cosmetic treatment of healthy skin and/or healthy keratin fibers damaged by UV rays or aggressive substances, particularly shampoos, substances used in hair coloring, and/or medications.

The invention also relates to the solid oil according to the invention for pharmaceutical use, preferably dermatological use, in preventing and/or treating winter xerosis, atopy, eczema, and/or alopecia.

The solid avocado oil according to the inventionis also incorporated as a substitute for mineral oils and lanolins.

The solid avocado oil according to the invention is also incorporated to combat the effects of actinic, chronological, and photo-induced aging. It can also be used as an anti-pollution and anti-allergenic film-forming agent or even as a protector of the skin microbiota.

In one embodiment, the cosmetic composition according to the invention is characterized in that it comprises from 0.1 to 95.0% by mass of the solid avocado oil according to the invention relative to the total mass of said composition.

In one embodiment, the cosmetic composition according to the invention is characterized in that it comprises from 1.0 to 90.0% by mass of the solid avocado oil according to the invention relative to the total mass of said composition.

In one embodiment, the cosmetic composition according to the invention is characterized in that it comprises from 2.0 to 75.0% by mass of the solid avocado oil according to the invention relative to the total mass of said composition.

In an emulsion intended for skin care, the solid avocado oil (CHA) is combined with a chemically transformed surfactant of plant origin selected from the group consisting of lecithins, alkyl betaines, ethoxylated fatty alcohols, esters of PEG and PPG, sucroesters, oxyethylenated sugars, sorbitol esters, alkyl polyglucosides (APG), monoglycerides, polyglycerol esters, alkyl sulfates, alkyl ether sulfates and sulfonates.

In an emulsion intended for skin care, the solid avocado oil (CHA) is combined with a thickening agent of chemically transformed plant origin selected from the group consisting of alginates, carrageenans, celluloses and modified starches, pectins, xanthan, guar, and arabic gums.

In an emulsion intended for skin care, the solid avocado oil (CHA) is combined with an emollient agent of chemically transformed plant origin selected from the group consisting of C₈ to C₁₈ fatty acid esters and alkanes.

In an emulsion intended for skin care, the solid avocado oil (CHA) is combined with a vegetable lipid or a fraction thereof selected from the group consisting of coconut, palm, palm kernel, olive, argan, soy, sunflower, rapeseed, rice bran, corn, cotton, wheat germ, sweet almond, avocado, safflower; hemp, walnuts, hazelnut, camelina, evening primrose, borage, blackcurrant, moringa, andiroba, jojoba, macadamia, Brazil nut, crambe, karanja, passionflower, castor, flax, pracaxi, buruti, baobab, calendula, pumpkin seed, sesame, grape seed, cuphea, babassu, marula, passionflower, apricot, chia, cranberry, perilla, raspberry, blueberry, shea butters, cocoa, mango, illipe, cupuacu, murumuru, kokum, sal.

In a shampoo formula, the solid avocado oil (CHA) is combined with a chemically transformed surfactant of plant origin selected from the group consisting of lecithins, alkyl betaines, ethoxylated fatty alcohols, PEG and PPG esters, sucroesters, oxyethylenated sugars, sorbitol esters, alkyl polyglucosides (APG), monoglycerides, polyglycerol esters, alkyl sulfates, alkyl ether sulfates, and sulfonates.

In a shampoo formula, the solid avocado oil (CHA) is combined with a vegetable lipid or a fraction thereof selected from the group consisting of the oils of coconut, palm, palm kernel, olive, argan, soy, sunflower, rapeseed, rice bran, corn, cotton, wheat germ, sweet almond, avocado, safflower; hemp, walnuts, hazelnut, camelina, evening primrose, borage, blackcurrant, moringa, andiroba, jojoba, macadamia, Brazil nut, crambe, karanja, passionflower, castor, flax, pracaxi, buruti, calendula, baobab, pumpkin seed, sesame, grape seed, cuphea, babassu, marula, passionflower, apricot, chia, cranberry, perilla, raspberry, blueberry, shea butters, cocoa, mango, illipe, cupuacu, murumuru, kokum, sal.

In a soap formula, the solid avocado oil (CHA) is combined with a vegetable lipid or a fraction thereof selected from the group consisting of the oils of coconut, palm, palm kernel, olive, argan, soy, sunflower, rapeseed, rice bran, corn, cotton, wheat germ, sweet almond, avocado, safflower; hemp, walnuts, hazelnut, camelina, evening primrose, borage, blackcurrant, moringa, andiroba, jojoba, macadamia, Brazil nut, crambe, karanja, passionflower, castor, flax, pracaxi, buruti, calendula, baobab, pumpkin seed, sesame, grape seed, cuphea, babassu, marula, passionflower, apricot, chia, cranberry, perilla, raspberry, blueberry, shea butters, cocoa, mango, illipe, cupuacu, murumuru, kokum, sal.

In a formula for lip care, the solid avocado oil (CHA) is combined with a wax of plant or animal origin selected from the group consisting of carnauba wax, candelilla wax, rice bran wax, sunflower wax, castor wax, jojoba wax, beeswax.

The invention also relates to a food composition which is characterized in that it comprises a solid avocado oil according to the invention.

The solid avocado oil according to the invention is intended to be incorporated into food products as an active ingredient or functional raw material.

Food products are understood to mean commonly consumed foods, food supplements, and clinical nutrition.

Thus, the solid avocado oil according to the invention can be advantageously incorporated into solid, liquid, or sprayable food compositions based on lipids and, more particularly, table oils, frying, margarines, spreads, pastries, ready-made meals, chocolate products, and confectionery.

The solid avocado oil can also be used in food compositions as a thickening agent, rheology modifier, film-forming agent, coating, lubricant, support for additives, flavors, and vitamins.

The solid avocado oil according to the invention can also be incorporated into solid or liquid food supplements, particularly in a water-dispersible form or even more advantageously in soft capsules.

As an active food ingredient, the solid avocado oil according to the invention is incorporated as a rebalancing agent for monounsaturated fatty acid intake or as a source of palmitic and palmitoleic acid.

In the field of food supplements for cosmetic purposes, also called cosmetofoods, or nutraceuticals, the solid avocado oil according to the invention is a lipid-replenishing, nutritive, hydrating agent that is reconstitutive of the hydrolipidic film but is also used as a promoter of epidermal lipid synthesis, as an agent for treating mature skin, winter xerosis, atopy, eczema, alopecia, dandruff, or even keratin fibers damaged by UV rays or aggressive substances, particularly selected from among shampoos, substances used in hair coloring, and/or medications.

The invention relates to the use of the solid oil according to the invention as an active ingredient in food supplements for cosmetic purposes, as a lipid-replenishing, nourishing, anti-dandruff, and/or moisturizing agent for healthy skin, particularly healthy scalp, and/or for the cosmetic treatment of healthy skin and/or healthy keratin fibers damaged by UV rays or aggressive substances, particularly shampoos, substances used in hair coloring, and/or medications.

In one embodiment, the food composition according to the invention is characterized in that it comprises from 0.1 to 98.0% by mass of the solid avocado oil according to the invention relative to the total mass of said composition.

In one embodiment, the food composition according to the invention is characterized in that it comprises from 1.0 to 90.0% by mass of the solid avocado oil according to the invention relative to the total mass of said composition.

In one embodiment, the food composition according to the invention is characterized in that it comprises from 2.0 to 75.0% by mass of the solid avocado oil according to the invention relative to the total mass of said composition.

In a food formulation, the solid avocado oil (CHA) is combined with a vegetable lipid or a fraction thereof selected from the group consisting of the oils of coconut, palm, palm kernel, olive, soy, sunflower, rapeseed, rice bran, corn, cotton, wheat germ, avocado, safflower; hemp, walnuts, hazelnut, camelina, evening primrose, borage, blackcurrant, flax, pumpkin seed, sesame, grape seed, chia, cranberry, perilla, shea butter, cocoa, mango, sal.

In a food supplement formulation, the solid avocado oil (CHA) is combined with a vitamin selected from the group consisting of vitamin A, C, E, D, K, B1, B2, B3, B5, B6, B8, B9, B12, niacin, folic acid, pantothenic acid, and biotin.

In a food supplement formulation, the solid avocado oil (CHA) is combined with a mineral salt selected from the group consisting of calcium, magnesium, iron, copper, iodine, zinc, manganese, potassium, selenium, chromium, molybdenum, fluoride, chlorine, and phosphorus.

In the examples, the temperature is expressed in degrees Celsius and the pressure is atmospheric pressure unless otherwise indicated.

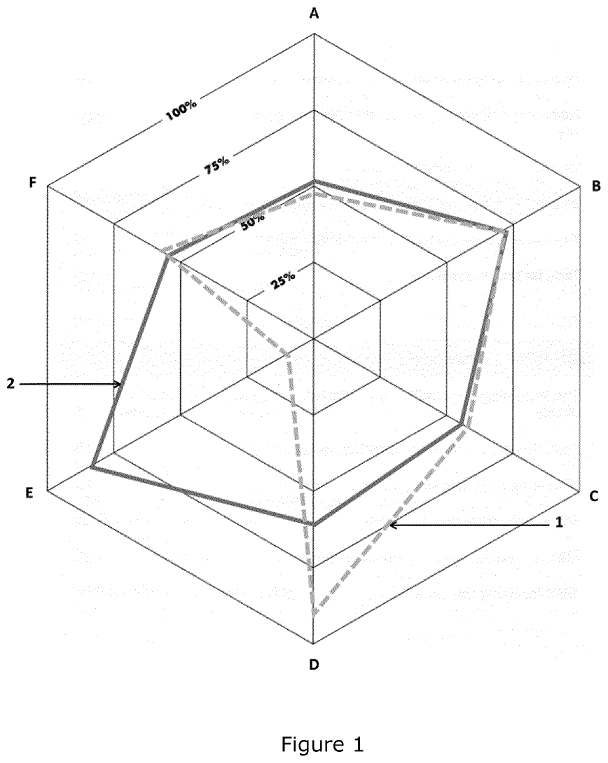

Legend for FIG. 1:
A: Rate of penetration
B: Smoothness on application
C: Richness on application
D: Sensation of nourished skin after 3 min
E: Lightening on application
F: Spreading
1: EMULSION A (solid avocado oil according to the invention)
2: EMULSION B (liquid paraffin)

### Example 1: Preparation of a Refined Avocado Oil and a Solid Avocado Oil

20 kg of fresh avocado of the Hass variety from Mexico were peeled and pitted in order to extract the pulp. The pulp was then crushed in a blender to obtain a homogeneous puree. In a stirred reactor, 20 liters of hot water at 60° C. were then added, and the mixture was stirred at 500 RPM for 40 minutes. The mixture was then centrifuged in order to separate the liquid phase, which consists of a water-oil emulsion. The operation was repeated three times. The liquid phases were then left to stand for 10 hours in order to separate the water and the oil by decantation. 1.81 kg of crude avocado oil were recovered. The oil obtained has an acidity of 1.5% in oleic acid equivalent.

In a stirred reactor, the crude oil was then heated under stirring to 75° C. in the presence of 500 g of a 0.2% citric acid solution for 30 minutes. 500 g of a 2% soda solution were then added. The mixture was maintained under stirring for 30 minutes and then separated by centrifugation in order to recover the oily phase. This oily phase was then washed 3 times with demineralized water (3 times 500 ml) until neutral in terms of pH. The oil obtained was finally dried under a vacuum of 100 mbar at 70° C. for 30 minutes. The resulting oil was then bleached in the presence of 2% bleaching earth (Tonsil 115 clay supplied by the company Clariant). Bleaching was carried out under stirring and under a vacuum of 50 mbar for 45 minutes. At the end of bleaching, the oil was filtered and recovered. The oil was then cooled to 10° C. under very slow stirring (20 RPM) for 48 hours and then filtered under nitrogen pressure. 1.128 g of a fluid oil (MG 1) and 262 g of filtration cake were then recovered in the form of a solid fat (MG 2).

The fluid oil MG 1 and the solid fat MG 2 were separately deodorized under a vacuum at 200° C. under a vacuum of 5 mbar and by continuous injection of water vapor (2 g of steam for 100 g of fat) for 90 minutes.

We then respectively obtain the refined fluid oil A1 and a solid oil at 20° C. according to the invention (solid avocado oil E1).

The oils A1 and the solid avocado oil E1 were analyzed (results shown in Tables 1, 2, and 3).

TABLE 1
Comparative analysis of the fatty acid distribution
of the oil A1 and the solid avocado oil E1.

| | ANALYTICAL CRITERIA | |
|---|---|---|
| | Solid avocado | Refined avocado |
| | oil E1 | oil A1 |
| | Appearance | |
| | Solid fat at 20° C. | Fluid oil at 20° C. |
| C16:0 - Palmitic acid | 22.1 | 15.3 |
| C16:1 - Palmitoleic acid | 7.9 | 7.5 |
| C18:0 - Stearic acid | 0.5 | 0.6 |
| C18:1 - Oleic acid | 56.5 | 64.9 |
| C18:2 - Linoleic acid | 11.2 | 10.6 |
| C18:3 - Linolenic acid | 1.1 | 0.7 |
| Total unsaturated fatty acids | 77.0 | 84.4 |
| Total monounsaturated fatty | 64.4 | 72.8 |
| acids | | |
| Total polyunsaturated fatty | 12.6 | 11.3 |
| acids | | |
| Total saturated fatty acids | 23.0 | 15.9 |
| Content of unsaponifiables | 0.83 | 0.81 |

### Comments:

The solid avocado oil E1 has a palmitic acid content which is increased by more than 44% compared to refined avocado oil obtained using a conventional process (oil A1);

The palmitoleic acid content of the solid avocado oil E1 was very slightly higher than that of the oil A1;

The monounsaturated fatty acid content remains relatively high compared to conventional avocado oil A1.

The content of polyunsaturated fatty acids remains relatively close in the oil A1 and the solid avocado oil E1;

The method for obtaining the solid avocado oil E1 does not alter the content of unsaponifiables (phytosterols and fat-soluble vitamins).

In conclusion, the solid avocado oil E1 is an ingredient that is rich in palmitic acid without notable alteration of the nutritional qualities of conventional avocado oil in terms of monounsaturated, polyunsaturated, and unsaponifiable fatty acids.

TABLE 2
Comparative analysis of the triglyceride composition
of the oil A1 and the solid avocado oil E1.

ANALYTICAL CRITERIA

| | |
|---|---|
| Solid avocado | Refined avocado |
| oil E1 | oil A1 |

Triglyceride composition, %

| | | |
|---|---|---|
| OLL - | ND | 5.0 |
| PLL | ND | 2.1 |
| PPoO | 5.0 | 3.4 |
| PoOO | 6.4 | <0.1) |
| POL | 7.6 | 13.2 |
| PP L | 1.9 | — |
| OOO | 22.2 | 29.0 |
| POO | 20.7 | 22.4 |
| PPO | 8.9 | 2.8 |
| PPP | 2.3 | 0.6 |
| PPS | <0.1 | — |
| OOS | 0.6 | 0.4 |
| OOL + LnPP | 10.8 | 22.9 |
| Miscellaneous | 1.4 | 3.4 |

In the nomenclature of triglycerides, radicals from fatty acids are abbreviated as follows:
L for linoleic acid,
O for oleic acid,
P for palmitic acid,
Po for palmitoleic acid, and
S for stearic acid.

Comments:
Solid avocado oil E1 and oil A1 have a completely different triglyceride composition;
The solid avocado oil E1 is rich in the triglycerides PPoO, PoOO, PPL, PPP, and PPL and, in particular, in triglycerides in which palmitic acid is in the sn2 position of the glycerol, i.e., the position that is most bioavailable orally.

The solid avocado oil E1 is also rich in palmitoleic acid vector triglycerides (PPoO and PoOO).

In conclusion, the solid avocado oil E1 is an ingredient that is rich in palmitic acid which is highly bioavailable through food.

TABLE 3
Comparative differential thermal analysis
of the oil A1 and the avocadosolid oil E1

| CRITERIA | |
|---|---|
| Solid avocado | Refined avocado |
| oil E1 | oil A1 |
| Appearance | |
| Solid fat | Fluid oil |
| at 20° C. | at 20° C. |

Estimated temperatures for end of melting and crystallization, ° C.

| | | |
|---|---|---|
| Temperature at end of total | −52.5 | <−80.0 |
| crystallization | | |
| Minimum/maximum | −36.0 to 43.5 | −75.0 to 22.0 |
| temperature at the majority | | |
| melting peak | | |
| Minimum/maximum | −53.1 to −3.4, | <−80.0 to −42.5 |
| temperature of the majority | | |
| crystallization peak | | |

### Comments:

The refined oil A1 and the solid avocado oil E1 have completely different melting and crystallization profiles, as evidenced by their respective end-of-crystallization temperatures as well as the minimum/maximum temperatures at the majority melting and crystallization peaks.

The solid avocado oil E1 is a solid fat at 20° C., unlike the refined avocado oil A1, which is almost entirely in liquid form at the same temperature.

In conclusion, on account of their melting and crystallization behavior, the oil A1 and the solid avocado oil E1 have fundamentally different cosmetic properties in terms of feel and emollience.

Likewise, their taste in the mouth and their food palatability (characteristic of the texture of foods that are pleasant to the palate, a property partly responsible for food pleasure in the mouth) are completely different. As such, solid avocado oil E1 is interesting for making spreads or margarines or even as a fat for chocolates and pastries.

### Example 2: “Body Care Cream” Cosmetic Composition Containing the Solid Avocado Oil

| Ingredients/INCI name | % by weight |
|---|---|
| Phase A | |
| Water | Q.s. 100 |
| Disodium EDTA | 0.02 |
| Allantoin | 0.1 |
| Glycerin | 10.0 |
| Beta glucan (0.4% solution) | 2.0 |
| Phase B | |
| Jojoba esters | 2.0 |
| C₁₄-C₂₂ alcohols (and) C₁₂-C₂₀ alkyl glucoside | 3.0 |
| Solid avocado oil E1 (according to the invention) | 6.0 |

### Example 3: Cosmetic Composition of Deodorant Stick Containing the Solid Avocado Oil

| Ingredients/INCI name | |
|---|---|
| Phase A | % by weight |
| Isododecane | 62.0 |
| Aluminum chlorohydrate | 21.0 |
| Cetyl alcohol | 5.0 |
| Solid avocado oil E1 (according to the invention) | 8.0 |
| Hydrogenated castor oil | 4.0 |

### Example 4: Composition of Food Supplement in Soft Capsules Containing Avocado Oil Extract

| Ingredient/additive | Content mg/capsule |
|---|---|
| Solid avocado oil E1 (according to the invention) | 35.0 |
| Refined macadamia oil | 10.0 |
| Vitamin E (alpha-tocopherol) | 5 |

### Example 5: Food Composition of Chocolate Spread Containing the Solid Avocado Oil

| Ingredient/additive | g/100 g |
|---|---|
| Cane sugar | 18.0 |
| Hazelnut paste | 15.0 |
| Solid avocado oil E1 (according to the invention) | 7.0 |
| Sunflower oil | 6.00 |
| Skimmed milk powder | 5.0 |
| Cocoa butter | 6.5 |
| Soy lecithin | 3.5 |
| Vanilla extract | 0.5 |
| Water | Q.s. 100 |

### Formula 6 (Comparative): Substitution of Conventional Avocado Oil in a Cosmetic Cream/Effect on the Amount of Thickening Agent

Two cosmetic formulations were prepared according to the same operating procedure, which consists of heating the fatty phase and the aqueous phase separately to 70° C. The aqueous phase was then added to the fatty phase under vigorous stirring. The pH was then adjusted to 6.5 by adding triethanolamine. After cooling with constant stirring to a temperature of 40%, the final addition of the perfume was carried out. The compositions of these 2 formulations are shown in the table below:

| | Formula A | Formula B |
|---|---|---|
| Ingredients (INCI name) | % by weight | % by weight |
| Oily phase | | |
| Isopropyl palmitate | 2.5 | 2.5 |
| Refined A1 Avocado Oil/Persea | 6.0 | — |
| americana (avocado) oil | | |
| Solid avocado oil E1 (according to the | — | 6.0 |
| invention) | | |
| Cetearyl alcohol (and) ceteareth-20* | 2.5 | — |
| Isopropyl palmitate | 2.5 | 2.5 |
| Phytosterols | 1.2 | 1.2 |
| Glyceryl stearate | 1.0 | 1.0 |
| Dimethicone** | 0.5 | 0.5 |
| Stearic acid* | 0.5 | — |
| Aqueous phase | | |
| Propylene glycol | 2.0 | 2.0 |
| Water | Q.s. 100 | Q.s. 100 |
| Additives | | |
| Triethanolamine | 0.2 | 0.2 |
| Perfume | q.s. | Q.s. |
| Viscosity under stress at 1 s⁻¹ and at | 8,250 | 8,100 |
| 25° C., cP | | |

*thickening agent
**viscosity 200/100 Cs

### Comments:

Note that formulas A and B, which have identical contents respectively of the refined avocado oil A1 and the solid avocado oil E1, differ only in terms of their respective contents of thickening ingredients (free and ethoxylated cetearyl alcohols, stearic acid). It can be seen, however, that their viscosity is substantially identical. This result demonstrates that the solid avocado oil E1 according to the invention has an intrinsic gelling activity and makes it possible to reduce the content of thickening agents.

### Example 7 (Comparative)—Substitution of Conventional Avocado Oil in Mayonnaise/Effect on the Quantity of Thickening Agent and Oxidation Resistance

Two formulations of mayonnaise were prepared according to the same operating procedure, which consists of emulsifying a first phase consisting of water, a thickening agent, and a flavoring using a homogenizer. The following were then added in four distinct and successive steps: the egg yolk, the sodium chloride and the preservative, the vinegar, and then finally the avocado vegetable oil. Preparation was carried out at a temperature of 20° C. and under a vacuum of 400 mbar. The two formulations were kept in the refrigerator at 5° C. and in contact with air for 15 days. The compositions and characteristics of these two formulations are shown in the table below:

| | Formula A | Formula B |
|---|---|---|
| Ingredients | % by weight | % by weight |
| Refined avocado oil A1 | 35.0 | — |
| Solid avocado oil E1 (according to | — | 35.0 |
| the invention) | | |
| Egg yolk | 8.1 | 8.1 |
| Salt (NaCl) | 1.4 | 1.4 |
| Vinegar | 4.0 | 4.0 |
| Guar gum (thickening agent) | 4.0 | 2.5 |
| Potassium benzoate (preservative) | 0.15 | 0.15 |
| Citral (flavor) | 0.03 | 0.03 |
| Water | 47.32 | 48.82 |
| Viscosity under stress at 25° C., Pas | 943 | 905 |
| Peroxide index, meq O₂/kg | 4.7 | 1.1 |

### Comments:

Note that formulas A and B, which have identical contents of refined avocado oil A1 and solid avocado oil E1, differ in their respective contents of thickening agent (guar gum). It can be seen, however, that their viscosity is substantially identical, whereas the guar gum content of formula B has been reduced by more than 35%. This result demonstrates that the solid avocado oil E1 according to the invention has intrinsic thickening activity and makes it possible to reduce the quantity of thickening agent.

What is more, the oxidation resistance of formula B was improved, since the peroxide index after 15 days of cold storage increases 4 times faster.

### Example 8 (Comparative)—Oxidation Resistance

The Rancimat method is an accelerated aging test. As the temperature in the reaction vessel steadily increases, air is forced through the sample. It measures the time required for product oxidation and defines resistance to or stability under oxidation (oxidative stability index, OSI). The refined avocado oil A1 and the solid avocado oil E1 according to the invention were the subject of this analysis according to the NF EN ISO 6886 standard. The results obtained are shown in the table below:

| | OSI time |
|---|---|
| Product tested | (Rancimat)*, h |
| Refined avocado oil A1 | 21.0 |
| Solid avocado oil E1 (according to the invention) | 26.5 |

*test carried out on a 3 g test specimen at 98° C. and under an injected air flow rate of 10 l/h.

### Comments:

Note that the solid avocado oil E1 according to the invention has an induction time that is significantly greater than that of the conventional avocado oil A1 and thus has a better oxidation resistance.

### Example 9 (Comparative)—the Solid Avocado Oil According to the Invention as a Source of High-Purity Palmitic Acid

In a stirred reactor equipped with a refrigerant, 200 g of the solid avocado oil E1 according to the invention were placed in the presence of 960 ml of 3.7M alcoholic potash and a few glass beads. The mixture was then brought to reflux (95° C.) for 4 hours. After cooling to room temperature, the medium was diluted with 450 ml of 4.0M sulfuric acid and refluxed at 95° C. for 6 hours. The mixture was then cooled slightly to 50° C. and then left to decant for 3 hours while maintaining the temperature at 50° C.

The oily phase was then separated from the heavy aqueous phase and then washed in a stirred reactor in the presence of 100 ml of hot water (60° C.). After decantation and recovery of the oily phase, the operation was repeated until the washing water was neutral. The oily phase was then dried under a vacuum (100 mbar) on a rotary evaporator. The oily phase was then stored at 12° C. for 24 hours in order to crystallize the saturated fatty acids. The mixture was then filtered through a Büchner filter, and the cake was washed 3 times with 100 ml of cold hexane (15° C.). The cake obtained was finally dried in an oven at 40° C. for 10 hours.

The procedure is the same with a refined palm oil.

The analysis of the fatty acid composition of the two cakes obtained is presented in the table below:

| | Fatty acids of | Fatty acids of |
|---|---|---|
| | the solid avocado | the refined palm |
| ANALYTICAL CRITERIA | oil E1, % | oil A1, % |
| C12:0 | ND* | 0.5 |
| C14:0 | ND* | 2.1 |
| C16:0 | 96.5 | 88.3 |
| C16:1 | ND* | ND* |
| C18:0 | 3.2 | 9.0 |
| C18:1 | 0.3 | 0.1 |
| C18:2 | ND* | ND* |
| C18:3 | ND* | ND* |

*Not detected

### Comments:

The palmitic acid content of the fatty acids obtained from the solid avocado oil E1 according to the invention is 96% and is significantly higher than that of the fatty acids obtained from refined palm oil. Therefore, the solid oil of avocado E1 enables palmitic acid of greater purity to be obtained.

### Example 10: Comparison of the Physicochemical and Organoleptic Stability of an Oil-In-Water (O/W) Cosmetic Emulsion Based on Mineral Oil, Refined Avocado Oil, or the Solid Avocado Oil According to the Invention

An emulsion A was prepared from the solid avocado oil according to the invention as prepared in Example 1 (solid avocado oil E1), an emulsion B comprising a paraffin oil (INCI: Paraffinum Liquidum) sold under the name HUILE DE PARAFFINE CODEX STANDARD Lby the company Aiglon™, and an emulsion C comprising a refined avocado oil, HUILE D'AVOCAT VIERGE and HUILE D'AVOCAT BIOLOGIQUE™ OIL™, from the company EMILE NOEL™, with all elements also being equal according to the following protocol.

In a glass beaker of suitable size, the solid avocado oil E1 according to the invention or the paraffin oil (Paraffinum Liquidum), or the refined avocado oil was mixed with the caprylic/capric triglyceride and the mixture of cetearyl alcohol and cetearyl glucoside in the proportions indicated in the table below in order to reach a total weight of 300 grams. The mixture was heated to 80° C. under magnetic stirring and constitutes phase 1. Meanwhile, in a glass beaker of suitable size, the water, glycerin, xanthan gum, and the phenoxyethanol and parabens mixture were mixed together. This mixture was heated to 80° C. under magnetic stirring and constitutes phase 2. When the two phases reached the temperature of 80° C., phase 1 was poured slowly into phase 2 under stirring in the rotor/stator at a speed of 3000 RPM. Stirring was maintained for 5 minutes, and then the mixture was cooled using a cold water bath with planetary stirring at a stirring speed of between 700 and 1200 RPM until the temperature of the emulsion obtained in this manner reached 30° C.

The compositions of emulsions A and B are reproduced below:

| | Emulsion A | Emulsion B | Emulsion C |
|---|---|---|---|
| Ingredients - (INCI name) | % by weight | % by weight | % by weight |
| Solid avocado oil E1 | 15.0 | / | / |
| (according to the invention) | | | |
| Paraffin oil | / | 15.0 | / |
| (Paraffinum Liquidum) | | | |
| Refined avocado oil | / | / | 15.0 |
| Caprylic/capric triglyceride | 5.0 | 5.0 | 5.0 |
| Cetearyl alcohol and | 3.0 | 3.0 | 3.0 |
| cetearyl glucoside | | | |
| Glycerin | 3.0 | 3.0 | 3.0 |
| Xanthan gum | 0.3 | 0.3 | 0.3 |
| Phenoxyethanol | 0.8 | 0.8 | 0.8 |
| (and) parabens | | | |
| Water | q.s. | q.s. | q.s. |

Emulsions A, B, and C were characterized organoleptically at D=0 and D+1 for appearance, color, and odor.

The pH measurement was carried out using a portable probe, a previously calibrated Milwaukee™-brand pH meter.

The viscosity measurement was carried out using a NEVVTRY NDJ-1-brand viscometer using rotor no. 4 and a speed of 30 revolutions/s.

The results are presented in the table below:

| Criterion/formula | Emulsion A | Emulsion B | Emulsion C |
|---|---|---|---|
| Appearance (D = 0) | Fluid emulsion | Fluid emulsion | Fluid emulsion |
| Color (D = 0) | White to | White to | Greenish |
| | off-white | off-white | yellow |
| Odor (D = 0) | Neutral | Neutral | Vegetal |
| pH (D + 1) | 7.0 | 7.0 | 7.0 |
| Viscosity at 20° C., | 12,000 mPas | 10,000 mPas | 12,000 mPas |
| (D + 1) | | | |

Note that the emulsions A and B have a similar appearance, pH, color, and odor. In emulsified form, the solid avocado oil according to the invention induces a slight increase in viscosity without modifying the fluidity of the emulsion.

At D=0, we note that emulsion C was very tinged with a very marked vegetable odor. The emulsion comprising the solid avocado oil according to the invention therefore offers a significant advantage compared to the emulsion comprising refined avocado oil, particularly for uses for cosmetic, pharmaceutical, or food formulations compared to refined avocado oil.

Emulsions A and B were then subjected to an accelerated aging test carried out in an oven at 45° C., under air, for 60 days.

The appearance, color, and odor were estimated organoleptically.

The measurement of the peroxide index (hereinafter PI) was carried out according to the method NM ISO 3960-2009.

The pH measurement was carried out using a portable probe, a previously calibrated Milwaukee™-brand pH meter.

The measurement of the viscosity index was carried out using a NEVVTRY NDJ-1-brand viscometer using rotor no. 4 and a speed of 30 revolutions/s.

All measurements were carried out on D+1, D+15, D+30, and D+60.

The results are presented in the table below:

| Days | D + 1 | D + 30 | D + 60 |
|---|---|---|---|
| EMULSION A | Appearance: fluid emulsion | Appearance: fluid emulsion | Appearance: fluid emulsion |
| (solid avocado | Color: white to off-white | Color: white to off-white | Color: white to off-white |
| oil according | Odor: neutral | Odor: neutral | Odor: neutral |
| to the invention) | pH: 7.0 | pH: 7.0 | pH: 7.0 |
| | Viscosity: 12,000 mPas | Viscosity: 12,000 mPas | Viscosity: 12,000 mPas |
| | PI < 0.2 meq O₂/kg | PI < 0.2 meq O₂/kg | PI < 0.2 meq O₂/kg |
| EMULSION B | Appearance: fluid emulsion | Appearance: fluid emulsion | Appearance: fluid emulsion |
| (paraffin oil) | Color: white to off-white | Color: white to off-white | Color: white to off-white |
| | Odor: neutral | Odor: neutral | Odor: neutral |
| | pH: 7.0 | pH: 7.0 | pH: 7.0 |
| | Viscosity: 10,000 mPas | Viscosity: 10,000 mPas | Viscosity: 10,000 mPas |
| | PI < 0.2 meq O₂/kg | PI < 0.2 meq O₂/kg | PI < 0.2 meq O₂/kg |

Note that the appearance, color, odor, pH, and viscosity of emulsions A and B remain constant over time. At 60 days, in emulsified form and versus mineral oil, the solid avocado oil according to the invention does not induce any increase in peroxide index, which demonstrates the oxidative chemical stability of the emulsion comprising the solid avocado oil according to the invention.

Unexpectedly, when in emulsified form, the solid avocado oil according to the invention has an oxidation resistance that is identical to that of mineral oil without the addition of an antioxidant to the formula.

### Example 11: Comparison of the Physicochemical and Organoleptic Stability of a Formulation of Cosmetic Lip Balm Based on Mineral Oil Versus the Solid Avocado Oil

Two formulas of lip balm were prepared, one from the solid avocado oil E1 according to the invention as prepared in Example 1 (hereinafter balm A), and the other from a commercial paraffin oil sold by the company Aiglon™ (hereinafter balm B).

In a glass beaker of suitable size, all of the ingredients were mixed in one by one in the proportions indicated in the table below in order to reach a total weight of 300 grams. The mixture was heated to 80° C. for 10 minutes under magnetic stirring. Stirring was then removed, and the mixture was allowed to cool to room temperature.

The compositions of the balms A and B are reproduced below:

| | Balm A | Balm B |
|---|---|---|
| Ingredients - (INCI name) | % by weight | % by weight |
| Cera alba (beeswax) | 32.0 | 32.0 |
| Brassica campestris seed oil | 30.0 | 30.0 |
| Solid avocado oil E1 | 30.0 | / |
| (according to the invention) | | |
| Paraffin oil (Paraffinum Liquidum) | / | 30.0 |
| Ricinus communis seed oil | 8.0 | 8.0 |

Balms A and B were characterized at D=0 on the basis of physicochemical, visual, and organoleptic criteria.

The results are shown in the table below:

| Criterion/formula | Balms A | Balms B |
|---|---|---|
| Appearance (D = 0) | Balm | Balm |
| Color (D = 0) | Egg shell | Egg shell |
| Odor (D = 0) | Characteristic at neutral | Characteristic at neutral |

It clearly appears that, when in the form of a balm and at high concentration (30% by weight), the solid avocado oil according to the invention does not induce a change in appearance, odor, or color compared to a paraffin mineral oil.

Balms A and B were then subjected to an accelerated aging test carried out in an oven at 45° C. under air for 60 days.

The appearance, color, and odor were estimated organoleptically.

The measurement of the peroxide index (hereinafter PI) was carried out according to the method NM ISO 3960-2009.

All measurements were carried out on D+1, D+30, and D+60.

The results are presented in the table below:

| Day | D + 1 | D + 15 | D + 30 | D + 60 |
|---|---|---|---|---|
| BALM A | Appearance: Balm | Appearance: compliant | Appearance: compliant | Appearance: compliant |
| | Color: Egg shell | Color: compliant | Color: compliant | Color: compliant |
| | Odor: Characteristic at neutral | Odor: compliant | Odor: compliant | Odor: compliant |
| | PI < 5.06 meq O₂/kg | PI < 3.1 meq O₂/kg | PI: 5.9 meq O₂/kg | PI: 6.4 meq O₂/kg |
| BALM B | Appearance: Balm | Appearance: compliant | Appearance: compliant | Appearance: compliant |
| | Color: Egg shell | Color: compliant | Color: compliant | Color: compliant |
| | Odor: Characteristic at neutral: | Odor: compliant | Odor: compliant | Odor: compliant |
| | PI: 5.58 meq O₂/kg | PI: 1.6 meq O₂/kg | PI: 5.6 meq O₂/kg | PI: 5.9 meq O₂/kg |

It clearly appears that, at 30 and 60 days, in 30% balm form and compared to mineral oil, the solid avocado oil does not induce any change in any of the criteria measured.

Unexpectedly, when in balm form and at a high concentration (30%), and with all other elements being equal, the solid avocado oil according to the invention has an oxidation resistance that is identical to that of mineral oil even without adding any idantsantiox to the formula.

Indeed, the loss of 1.6 peroxide index points after 60 days and at 45° C. is extremely moderate given the drastic conditions of the accelerated aging test. Indeed, those skilled in the art would have considered a loss of at least 5 points on the peroxide index would have indicated an average oxidation resistance which, in any case, was significantly different from the oxidation resistance of balm based on paraffin oil.

Furthermore, it should be noted that the most easily oxidizable oil—rapeseed oil (INCI name Brassica campestris seed oil)—has a high content (i.e., 30% by weight) in the two balm formulations. Also, this oil, which is known for its poor oxidation resistance, was largely responsible for the increase in the peroxide index of the two balms. Consequently, in the absence of an antioxidant, the solid avocado oil according to the invention reinforces the protection of rapeseed oil against oxidation within the formula in a manner that is almost identical to paraffin oil.

### Example 12: Comparison of the Sensory Properties of an Oil-In-Water (O/W) Cosmetic Emulsion Based on Mineral Oil or on the Solid Avocado Oil According to the Invention

A sensory analysis was carried out on a panel of 20 women with emulsions A and B as prepared in Example 10, in order to compare the sensory performances of an emulsion comprising a solid avocado oil according to the invention and a conventional emulsion of paraffin oil.

The people recruited for this study were adults aged 25 to 45, all skin types combined. Each person responded individually to the study, rating each criterion from 1 to 5, without having received any information concerning the formulas to be evaluated (single-blind test).

The evaluation criteria as well as the results are shown in FIG. 1.

It can be seen that the panel did not perceive any significant sensory difference between the 2 formulas in terms of spreading, rate of penetration, smoothness on spreading, and richness on spreading. On these 4 criteria, emulsion A comprising the solid avocado oil according to the invention yielded results equivalent to those of emulsion B comprising paraffin oil.

The panel perceived a clear difference with regard to lightening on application, a characteristic that we seek to avoid in cosmetic formulation. On this point, emulsion A comprising the solid avocado oil according to the invention yields better results, since the latter hardly lathers up at all on application, unlike emulsion B comprising paraffin oil.

The panel perceived a significant difference with regard to the sensation of nourished skin after 3 minutes in favor of emulsion A comprising the solid avocado oil according to the invention.

All other elements being equal, the cosmetic compositions in the form of an emulsion comprising the solid avocado oil according to the invention therefore present a certain sensory advantage for the user compared to the cosmetic compositions comprising a paraffin oil.

1. A solid avocado oil, wherein its palmitic acid content relative to total fatty acids is between 15 and 35% and that its complete melting point is within a range of from 20 to 50° C., the oil of the avocado being obtained from avocado of a variety selected from the list consisting of Hass, Fuerte, Bacon, Pinkerton, Ettinger, Lula, Ryan, Barker, and/or Peruano.

2. The solid avocado oil according to claim 1, wherein it is obtained from Hass-variety avocado.

3. The solid avocado oil according to claim 1, wherein it is not obtained from avocado varieties of Malaysian origin.

4. The solid avocado oil according to claim 1, wherein it has a pour point as determined by the ASTM D 97 method of between 8 and 15° C.

5. The solid avocado oil according to claim 1, wherein its content of triglycerides in which palmitic acid is in position 2 of the glycerol is greater than or equal to 10% relative to the total triglyceride content.

6. The solid avocado oil according to claim 1, wherein its relative content of palmitoleic acid vector triglycerides is greater than or equal to 4.5% relative to the total triglyceride content.

7. The solid avocado oil according to claim 1, wherein its relative triolein content is less than or equal to 25%, relative to the total triglyceride content.

8. The solid avocado oil according to claim 1, wherein it is obtained after at least one winterization step.

9. A cosmetic or pharmaceutical composition, wherein it comprises, in a cosmetically or pharmaceutically acceptable vehicle, a solid avocado oil according to claim 1.

10. The composition as set forth in claim 9, wherein it further comprises an active ingredient.

11. Food composition, wherein it further comprises a solid avocado oil according to claim 1.

12. A method for obtaining a solid avocado oil according to claim 1, wherein it comprises the following steps:
fresh or dehydrated fruit,
and that it comprises at least the steps of:
grinding, extraction of crude oil,
eliminating a fluid oil by winterization and filtration,
obtaining a solid fat,
deodorization.

13. A method for obtaining a solid avocado oil according to claim 1, wherein it is carried out on:
crude avocado oils,
and that it comprises at least the steps of:
eliminating a fluid oil by winterization and filtration,
obtaining a solid fat,
deodorization.

14. A method for obtaining a solid avocado oil according to claim 1, wherein it is carried out on:
co-products from the refining or dewaxing of crude avocado oils,
and that it comprises at least the steps of:
deodorization.

15. The method according to claim 12, wherein, before the deodorization step, it comprises at least one of the following steps:
water degumming
chemical neutralization
physical neutralization
bleaching
cold crystallization and isolation of the precipitated fraction.

16. The method according to claim 11, wherein it further comprises a step of fractionation by hydrolysis.
